(19) United States
(12) Patent Application Publication
(10) Pub. No.: US 2019/0099347 A1
(43) Pub. Date: Apr. 4, 2019

(54) Bodily Cream

(71) Applicant: Clark Spencer, Aurora, CO (US)

(72) Inventor: Clark Spencer, Aurora, CO (US)

(21) Appl. No.: 15/724,680

(22) Filed: Oct. 4, 2017

(51) Int. Cl. A61K 8/67; A61Q 19/08; A61K 8/41; A61K 8/65; A61K 8/46; A61K 8/73; A61K 8/64

(52) U.S. Cl. CPC A61K 8/671; A61Q 19/08; A61K 8/676; A61K 8/41; A61K 8/64; A61K 8/678; A61K 8/46; A61K 8/735; A61K 8/65

(57) ABSTRACT

A bodily cream includes a base cream that is selectively applied to skin. A Matrixyl cream is mixed with the base cream to enhance collagen synthesis, collagen IV synthesis and hyaluronic acid synthesis in the skin. A Matrixyl 3000 cream is mixed with the base cream to reduce to the appearance of facial wrinkles. A Matrixyl Synthe-6 cream is mixed with the base cream to reduce forehead wrinkles and crow's feet wrinkles. Additionally, a Matrixyl 3000 peptide serum is mixed with the base cream to reduce the appearance of facial wrinkles. Finally, a Matrixyl Synthe-6 peptide serum is mixed with the base cream to reduce forehead wrinkles and crow's feet wrinkles.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to compounds and more particularly pertains to a new compound for reducing wrinkles in human skin.

### BRIEF SUMMARY OF THE INVENTION

An embodiment of the disclosure meets the needs presented above by generally comprising a base cream that is selectively applied to skin. A Matrixyl cream is mixed with the base cream to enhance collagen synthesis, collagen IV synthesis and hyaluronic acid synthesis in the skin. A Matrixyl 3000 cream is mixed with the base cream to reduce to the appearance of facial wrinkles. A Matrixyl Synthe-6 cream is mixed with the base cream to reduce forehead wrinkles and crow's feet wrinkles. Additionally, a Matrixyl 3000 peptide serum is mixed with the base cream to reduce the appearance of facial wrinkles. Finally, a Matrixyl Synthe-6 peptide serum is mixed with the base cream to reduce forehead wrinkles and crow's feet wrinkles.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### DETAILED DESCRIPTION OF THE INVENTION

A new compound embodying the principles and concepts of an embodiment of the disclosure and generally designated by the reference numeral 10 will be described.

The bodily cream 10 generally comprises a base cream that is selectively applied to skin on a human body. The base cream comprises a Retinol cream, a vitamin C cream, a DMAE cream, and a collagen cream. Each of Retinol cream, the vitamin C cream, the DMAE cream comprise 14.2% of the base cream. The collagen cream comprises 7.4% of the base cream. The base cream further comprises a vitamin E cream, a MSM cream, a Hyaluronic cream and an Emollient body lotion. Each of the vitamin E cream, the MSM cream and the Hyaluronic cream comprise 14.2% of the base cream. The Emollient body lotion comprises 7.4% of the base cream.

The Retinol cream may include purified water, propylene glycol, coconut oil, glyceryl stearate, mineral oil, stearic acid, cetyl esters, propylene glycol (and) diazolidinyl urea and methryparaben (and) propylparaben. The Retinol cream may further include triethanolamine, isopropyl myristate, retinyl palmitate, cetyl alcohol and dimethicone. Continuing, the Retinol cream may include fragrance, tocopherol, tocopheryl acetate, disodium EDTA and yellow 10.

The vitamin C cream may include water, glyceryl stearate, carthamux tinctorius seed oil, sorbitol, cetyl alcohol, stearic acid and butyrospermum parkii. The vitamin C cream may further include triethanolamine, caprylic/capric triglyceride, squalene, methylparaben, quaternium-15 and proplyparaben. Additionally, the vitamin C cream may include fragrance, ascorbyl palmitate, panthenol, ethylhexyl dimethyl PABA, tocopheryl acetate and cucumis sativus fruit extract. The vitamin C cream may include chamomilla recutita flower extract, alo barbadensis leaf extract and spiraea ulmaria flower extract.

The DMAE cream may include purified water, cetearyl alcohol (and) polysorbate 60, caprylic/capric triglyceride, prunus amygdalus dulcis oil and propylene glycol. The DMAE cream may include soluble collagen, propylene glycol (and) diazolindinyl urea, methylparaben (and) proplylparaben, and triethanolamine. Continuing, the DMAE cream may include sodium hyaluronate, DMAE bitarate and carbomer.

The Collagen cream may include deionized water, glycerine, caprylic/capric triglycerides, propylene glycol, glyceryl monostearate and glyceryl stearate. The Collagen cream may include PEG-100 stearate, stearic acid, isopropyl palmitate, cetyl alcohol, and ceteareth-20. Additionally, the Collagen cream may include cetearyl alcohol, hydrolyzed collagen, cetyl palmitate, shea butter, panthenol and vitamin E. The Collagen cream may additionally include dimethicone crosspolymer, dimethicone, methyl paraben, propyl paraben, disodium EDTA, diazolidinyl urea, triethanolamine and fragrance.

The vitamin E cream may include purified water, tocopheryl acetate, petrolatum, C12-15 alkyl benzoate, cetearyl alcohol (and) ceteareth-20 and glyceryl stearate. The vitamin E cream may include cetyl esters, stearic acid, mineral oil, phenoxyethanol (and) ethylexyl-glycerine and trethanolamine. Continuing, the vitamin E cream may include sodium magnesium silicate, polysorbate 80 and triticum bulgare germ oil.

The MSM cream may include purified water, dimethyl sofone, glycerine, glyceryl stearate, stearic acid, cetearly alcohol, ploysorbate 60 and helianthus annuus seed oil. The MSM cream may include cetyl alchohol, simmondsia chinesis seed oil, prunus amygdalus dulcis oil and ethylhexylglycerine (and) phenoxyethanol, polysorbate 80. Further, the MSM cream may include triethanolamine, acrylates/c10-30 alkyl acrylate crosspolymer, sodium EDTA, panthenol, hydrolyzed collagen, tocopheryl acetate and rosmarinus officinalis leaf oil. The MSM cream may include methyl salicylate (and) lavandula angustifolia oil, chamomile recutita flower oil (and) thymus vulgaris oil (and) citrus aurantium dulcis flower oil. Additionally, the MSM cream may include aloe barbadensis leaf juice, ginko biloba leaf extract, lecithin, retinyl palmitate and cholecalciferol.

The hyaluronic acid cream may include water, caprylic/capric triglycerides, emulsifying wax NF, glycerine, isopropyl myristate and stearic acid. The Hyaluronic acid cream may include helianthus annuss seed oil, butylene glycol (and) calluna vulgaris extract and sodium hyaluronate. Additionally, The hyaluronic cream may include tocopheryl acetate, glyceryl stearate, and phenoxyethanol (and) chlorphenesin (and) glycerine (and) sorbic acid. The hyaluronic acid cream may include dimethicone, cetyl alcohol, polysorbate 80, perfume, triethanolamine and acrylates/C10-30 alkyl acrylate crosspolymer. Moreover, the hyaluronic acid cream may include sodium magnesium silicate, tetrasodium EDTA, tocopherol and bisabolol. Additionally, the base cream may include 5 ml of DMAE serum and 20 ml of pure African Boabab oil.

A Matrixyl cream is mixed with the base cream to enhance collagen synthesis, collagen IV synthesis and hyaluronic acid synthesis in the skin. According to Peptidesciences.com the Matrixyl cream was shown to increase collagen synthesis up to 117%, collagen IV synthesis up to 327% and hyaluronic acid synthesis up to 267%. The Matrixyl cream may include deionized water, witch hazel extract, glycerol stearate, PEG-100 stearate, glycerin, butylene glycol, and carbomer. The Matrixyl cream may include polysorbate 20, palmitoyl pentapeptide-4, shea butter, and caprylic/capric triglyceride. Additionally, the Matrixyl cream may include phenoxyehthanol, caprylyl glycol, sorbic acid, glyceryl polymethacrylate and PEG 8 oligopeptide. The Matrixyl cream may include ammonium acryloyldimethyltaurate VP copolymer, ceateryl olivate, sorbitan olivate and PPG-12 SMDI/copolymer. Continuing, the Matrixyl cream may include sodium hyaluronate, tocopheryl acetate, disodium EDTA, xanthan gum, peppermint oil, green tea extract and tetrahexyldecyl ascorbate.

A Matrixyl 3000 cream is mixed with the base cream to reduce to the appearance of facial wrinkles. Matrixylinside.com has shown that Matrixyl 3000 has reduced deep wrinkles by up to 30% in a two month clinical study. A Matrixyl Synthe-6 cream is mixed with the base cream to reduce forehead wrinkles and crow's feet wrinkles. Matrixylinside.com has shown that Matrixyl Synthe-6 cream visibly reduced forehead wrinkles and crow's feet wrinkles in two month clinical trials.

Each of the Matrixyl 3000 cream and the Matrixyl Synthe-6 cream may include capric/caprylic triglyceride, aloe barbadensis leaf extract, glyceryl stearate SE, stearic acid, cetearyl alcohol and glycerine. Each of the Matrix 3000 cream and Matrixyl Synthe-6 creams may include simmondsia chinensis seed oil, prunus dulcis oil, shea butter, grape seed oil, soy peptide, and hydrolyzed marine collagen. Additionally, each of the Matrixyl 3000 and Matrixyl Synthe-6 creams may include retinyl palmitate, tocopherol acetate, camellia sinensis leaf extract, ceramide-3, malus domestica fruit cell culture extract and sodium hyaluronate. Each of the Matrixyl 3000 and Matrixyl Synthe-6 creams may include orange extract, schlerotium gum, phenoxyethanol, caprylyl glycol and potassium sorbate.

A Matrixyl 3000 peptide serum is mixed with the base cream to reduce the appearance of facial wrinkles. A Matrixyl Synthe-6 peptide serum is mixed with the base cream to reduce forehead wrinkles and crow's feet wrinkles. Each of The Matrixyl 3000 peptide serum and the Matrixyl Synthe-6 peptide serum may include hyaluronic acid, argireline, trylagen, polysorbate 20, glycerine, butylene glycol carbomer, phenoxyethanol and ethylhexylglycerine.

The Matrixyl cream comprises 2.0 fluid ounces of a total of 35.0 fluid ounces of a mixture of the base cream, the Matrixyl cream, the Matrixyl 3000 cream, the Matrixyl Synthe-6 cream, the Matrixyl 3000 peptide serum and the Matrixyl Synthe-6 peptide serum. Each of the Matrixyl 3000 cream and the Matrixyl Synthe-6 cream comprise 4.0 fluid ounces of the mixture. Finally, Each of the Matrixyl 3000 peptide serum and the Matrixyl Synthe-6 peptide serum comprise 5 ml of the mixture.

In use, the mixture is applied to skin on face, hands, knees, elbows, stretch marks and blemishes with fingers. The mixture is rubbed into the skin when the skin has been freshly washed. Additionally, the mixture is applied prior to applying makeup or other skin products. Thus, the mixture enhances health of the skin and reduces the appearances of wrinkles on the skin.

With respect to the above description then, it is to be realized that the optimum dimensional relationships for the parts of an embodiment enabled by the disclosure, to include variations in size, materials, shape, form, function and manner of operation, compound and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A bodily cream compound being configured to be applied to skin thereby enhancing health and appearance of the skin, said cream comprising:
a base cream being configured to be applied to skin;
a Matrixyl cream being mixed with said base cream wherein said Matrixyl cream is configured to enhance collagen synthesis, collagen IV synthesis and hyaluronic acid synthesis in the skin;
a Matrixyl 3000 cream being mixed with said base cream wherein said Matrixyl 3000 cream is configured to reduce to the appearance of facial wrinkles;
a Matrixyl Synthe-6 cream being mixed with said base cream wherein said Matrixyl Synthe-6 cream is configured to reduce forehead wrinkles and crow's feet wrinkles;
a Matrixyl 3000 peptide serum being mixed with said base cream wherein said Matrixyl 3000 peptide serum is configured to reduce the appearance of facial wrinkles; and
a Matrixyl Synthe-6 peptide serum being mixed with said base cream wherein said Matrixyl Synthe-6 peptide serum is configured to reduce forehead wrinkles and crow's feet wrinkles.

2. The compound according to claim x, wherein said base cream comprises a Retinol cream comprising 14.2% of said base cream.

3. The compound according to claim 2, wherein said base cream comprises a vitamin C cream comprising 14.2% of said base cream.

4. The compound according to claim 3, wherein said base cream comprises a DMAE cream comprising 14.2% of said base cream.

5. The compound according to claim 4, wherein said base cream comprises a Collagen cream comprising 7.4% of said base cream.

6. The compound according to claim 5, wherein said base cream comprises a vitamin E cream comprising 14.2% of said base cream.

7. The compound according to claim 6, wherein said base cream comprises a MSM cream comprising 14.2% of said base cream.

8. The compound according to claim 7, wherein said base cream comprises a Hyaluronic cream comprising 14.2% of said base cream.

9. The compound according to claim 8, wherein said base cream comprises an Emollient body lotion comprising 7.4% of said base cream.

10. The compound according to claim 1, wherein said Matrixyl cream comprises 2.0 fluid ounces of a total of 35.0 fluid ounces of a mixture of said base cream, said Matrixyl cream, said Matrixyl 3000 cream, said Matrixyl Synthe-6 cream, said Matrixyl 3000 peptide serum and said Matrixyl Synthe-6 peptide serum.

11. The compound according to claim 10, wherein said Matrixyl 3000 cream comprises 4.0 fluid ounces of said mixture.

12. The compound according to claim 11, wherein said Matrixyl Synthe-6 cream comprises 4 fluid ounces of said mixture.

13. The compound according to claim 12, wherein said Matrixyl 3000 peptide serum comprises 5 ml of said mixture.

14. The compound according to claim 13, wherein said Matrixyl Synthe-6 peptide serum comprises 5 ml of said mixture.

15. A bodily cream compound being configured to be applied to skin thereby enhancing health and appearance of the skin, said cream comprising:
a base cream being configured to be applied to skin, said base cream comprising:
a Retinol cream comprising 14.2% of said base cream,
a vitamin C cream comprising 14.2% of said base cream,
a DMAE cream comprising 14.2% of said base cream,
a Collagen cream comprising 7.4% of said base cream,
a vitamin E cream comprising 14.2% of said base cream,
a MSM cream comprising 14.2% of said base cream,
a Hyaluronic cream comprising 14.2% of said base cream, and
Emollient body lotion comprising 7.4% of said base cream;
a Matrixyl cream being mixed with said base cream wherein said Matrixyl cream is configured to enhance collagen synthesis, collagen IV synthesis and hyaluronic acid synthesis in the skin;
a Matrixyl 3000 cream being mixed with said base cream wherein said Matrixyl 3000 cream is configured to reduce to the appearance of facial wrinkles;
a Matrixyl Synthe-6 cream being mixed with said base cream wherein said Matrixyl Synthe-6 cream is configured to reduce forehead wrinkles and crow's feet wrinkles;
a Matrixyl 3000 peptide serum being mixed with said base cream wherein said Matrixyl 3000 peptide serum is configured to reduce the appearance of facial wrinkles; and
a Matrixyl Synthe-6 peptide serum being mixed with said base cream wherein said Matrixyl Synthe-6 peptide serum is configured to reduce forehead wrinkles and crow's feet wrinkles; and
said Matrixyl cream comprising 2.0 fluid ounces of a total of 35.0 fluid ounces of a mixture of said base cream, said Matrixyl cream, said Matrixyl 3000 cream, said Matrixyl Synthe-6 cream, said Matrixyl 3000 peptide serum and said Matrixyl Synthe-6 peptide serum, each of said Matrixyl 3000 peptide serum and said Matrixyl Synthe-6 peptide serum comprising 5 ml of said mixture.
